(19) United States
(12) Patent Application Publication
(10) Pub. No.: US 2026/0358346 A1
(43) Pub. Date: Nov. 12, 2026

(54) Methods for Improved Sequence Coverage in Analysis of Large Polypeptides

(71) Applicant: DH Technologies Development Pte. Ltd., Singapore (SG)

(72) Inventors: Wen JIN, Concord (CA); Haichuan LIU, Redwood City, CA (US); Pavel RYUMIN, Concord (CA); Yuzhuo ZHANG, Redwood City, CA (US)

(21) Appl. No.: 19/152,028

(22) PCT Filed: Jan. 31, 2024
(86) PCT No.: PCT/IB2024/050891

§ 371 Date: Jul. 30, 2025

Related U.S. Application Data
(60) Provisional application No. 63/442,645, filed on Feb. 1, 2023.
(60) Provisional application No. 63/466,880, filed on May 16, 2023.

(51) Int. Cl. G01N 33/68; G01N 1/40

(52) U.S. Cl. CPC G01N 33/6818; G01N 1/4044; G01N 33/6848; G01N 2333/70503

(57) ABSTRACT

In some embodiments, a method for improved sequence coverage of a polypeptide comprises removing interchain disulfide bonds and retaining essentially all intrachain disulfide bonds of a first sample and performing a first mass spectrometry (MS) analysis of the first sample to determine a first amino acid sequence, removing interchain disulfide bonds and intrachain disulfide bonds of a second sample and performing a second MS analysis to determine a second amino acid sequence, and combining the first and second sequence to determine a combined amino acid sequence of the polypeptide.

### RELATED US APPLICATIONS

The present application claims the benefit of priority from U.S. Provisional Applications Nos. 63/442,645 and 63/466,880, filed on Feb. 1, 2023 and May 16, 2023, respectively, the entire contents of which are hereby incorporated by reference.

### TECHNICAL FIELD

The present disclosure relates to polypeptides sequence measurement in mass spectrometry, and in particular, to improved sequence coverage in analysis of large polypeptides.

### BACKGROUND

Mass spectrometry (MS) is an analytical technique for determining the elemental composition of a substance. Specifically, MS may measure a mass-to-charge ratio (m/z) of ions generated from a test substance. MS may be used to identify unknown compounds, to determine isotopic composition of elements in a molecule, to determine the structure of a particular compound by observing its fragmentation, and to quantify the amount of a particular compound in a sample. Mass spectrometers detect ions and as such, a test sample must be converted to an ionic form during mass analysis.

When a polypeptide goes through an MS system and undergoes fragmentation, different values of fragment m/z of a polypeptide may be measured as different peaks in a spectrum. Such peaks may then be interpreted and attributed to specific fragment ions originating from the polypeptide. In this process the sequence of the polypeptide may be determined.

Conventionally large polypeptides are often enzymatically digested into smaller peptides before the sequence analysis by mass spectrometry. This method of analysis is known as the bottom-up approach. This approach generally requires less fragments for sequence reconstruction, which are therefore easier to analyze by mass spectrometry. However, such an approach involves the preliminary additional step of enzymatically digesting the polypeptides, which may take time and may also be prone to artifact

### INTRODUCTION

Alternative approaches to the bottom-up approach include sequencing the large polypeptide directly (top-down approach) or performing a limited proteolytic digestion step (middle-down approach) without the risk of introducing an artifact. All these approaches, however, reveal shortcomings for analysis of large polypeptides, such as proteins, that may have a complex structure with primary, and optionally secondary, tertiary, and even higher levels of structures often connected via mechanisms such as interchain and intrachain disulfide bonds. Therefore, it is important to devise a method to optimally sequence such molecules in a novel way for improved sequence coverage.

### SUMMARY

Some embodiments relate to a method for sequencing a polypeptide, the method including: removing one or more interchain disulfide bonds and retaining essentially all intrachain disulfide bonds of a first sample of the polypeptide to generate first set of subunits; performing a first mass spectrometry (MS) analysis of the first set of subunits to determine a first amino acid sequence of the polypeptide, removing one or more interchain disulfide bonds and one or more intrachain disulfide bonds of a second sample of the polypeptide to generate second set of subunits; performing a second MS analysis of the second set of subunits to determine a second amino acid sequence of the polypeptide; and combining the first amino acid sequence and the second amino acid sequence to determine a combined amino acid sequence of the polypeptide.

Some embodiments relate to a method, wherein a fragmentation process is utilized to determine an amino acid sequence.

Some embodiments relate to a method, wherein the fragmentation scheme is selected from one of electron activated dissociation and electron capture dissociation, ultraviolet photo-dissociation, and electron transfer dissociation.

Some embodiments relate to a method, wherein removing one or more interchain disulfide bonds includes removing essentially all interchain disulfide bonds.

Some embodiments relate to a method, wherein the second sample is the same as the first sample.

Some embodiments relate to a method, wherein the first sample and the second sample are subsamples of the same sample.

Some embodiments relate to a method, wherein the first sequence contains at least a section from a middle portion of the polypeptide.

Some embodiments relate to a method, wherein the second sequence contains at least a portion enclosed by intrachain disulfide bond.

Some embodiments relate to a method, wherein the polypeptide is a protein.

Some embodiments relate to a method, wherein the polypeptide is an antibody.

Some embodiments relate to a method, wherein the antibody is one of a monoclonal antibody (mAb), a bispecific antibody (bsAb), and an antibody drug conjugate (ADC).

Some embodiments relate to a method, wherein tandem mass spectrometry analysis is used for MS analysis.

Some embodiments relate to a method, wherein removing a disulfide bond is performed by using a chemical agent.

Some embodiments relate to a method, wherein the chemical agent is selected from a group consisting of DTT (Dithiothreitol), TCEP (tris (2-carboxyethyl) phosphine) or mercaptoethanol.

Some embodiments relate to a method, wherein removing a disulfide bond is performed electrochemically.

Some embodiments relate to a method wherein an enzymatic digestion of the sample is performed prior to removing a disulfide bond.

Some embodiments relate to a method, wherein an enzyme performing the enzymatic digestion is one of IdeS, IgdE, IdeZ, Kgp, and SpeB.

Some embodiments relate to a method, wherein removing one or more interchain disulfide bonds of the first sample includes removing essentially all interchain disulfide bonds.

Some embodiments relate to a method, wherein removing one or more interchain disulfide bonds and one or more intrachain disulfide bonds of the second sample includes removing essentially all interchain and intrachain disulfide bonds.

Some embodiments relate to a method for sequencing a polypeptide, the method including: utilizing a chemical reaction to link a portion of a sample of the polypeptide; and performing MS analysis of the sample to determine an amino acid sequence of the polypeptide.

Some embodiments relate to a method, further performing MS analysis of a second sample of the polypeptide without utilizing the chemical reaction to determine a second amino acid sequence of the polypeptide and combining the amino acid sequence and the second amino acid sequence to determine a combined amino acid sequence of the polypeptide.

Some embodiments relate to a method, wherein the chemical reaction is a reaction with the cross-linking agent.

Some embodiments relate to a method, wherein the chemical reaction is a reaction with a metal cation.

Some embodiments relate to a method for sequencing a biopolymer, the method including: utilizing physicochemical interaction to link a portion of a sample of the biopolymer; performing MS analysis of the sample to determine an amino acid sequence of the biopolymer.

Some embodiments relate to a method, further performing MS analysis of a second sample of the biopolymer without utilizing physicochemical interaction to determine a second amino acid sequence of the polypeptide and combining the amino acid sequence and the second amino acid sequence to determine a combined amino acid sequence of the polypeptide.

Some embodiments relate to a method, wherein the physicochemical interaction is an ion-ion interaction.

Some embodiments relate to a method, wherein the ion-ion interaction is the result of an ion-ion reaction.

Some embodiments relate to a method, wherein the ion-ion reaction is performed in gas phase.

Some embodiments relate to a method, wherein a reactant polypeptide is a polypeptide of an opposite charge of an analyte biopolymer.

Some embodiments relate to a method, wherein the biopolymer is one of a polypeptide and a polynucleotide.

Some embodiments relate to a method, wherein the subunit polypeptide mass is above 20 kDa.

Some embodiments relate to a method and 24, wherein an analyte polypeptide mass is above 20 kDa.

Some embodiments relate to a method and 32, wherein the sequence contains at least a portion of the middle of the protein.

Some embodiments relate to a method for sequencing a middle portion of a polypeptide, the method including: removing essentially all interchain disulfide bonds and retaining essentially all intrachain disulfide bonds of a sample of the polypeptide to generate subunits, performing mass spectrometry (MS) analysis of the subunits to determine an amino acid sequence of the middle portion of the polypeptide.

Some embodiments relate to a method wherein the middle portion of the polypeptide spans a portion further than 50 amino acids from the polypeptide termini.

Some embodiments relate to a method wherein the middle portion of the polypeptide spans a portion further than 70 amino acids from the polypeptide termini.

Some embodiments relate to a method wherein the middle portion of the polypeptide spans a portion further than 100 amino acids from the polypeptide termini.

Some embodiments relate to a method for sequencing a polypeptide from a sample, the method comprising: removing one or more interchain disulfide bonds and retaining essentially all intrachain disulfide bonds of a first portion of a sample of the polypeptide to generate first set of subunits; removing one or more interchain disulfide bonds and one or more intrachain disulfide bonds of a second portion of a sample of the polypeptide to generate second set of subunits; separating the first set of subunits from the second set of subunits; performing a mass spectrometry (MS) analysis of the first set of subunits to determine a first amino acid sequence of the polypeptide; performing a second MS analysis of the second set of subunits to determine a second amino acid sequence of the polypeptide; and combining the first amino acid sequence and the second amino acid sequence to determine a combined amino acid sequence of the polypeptide.

Further understanding of various aspects of the embodiments may be obtained by reference to the following detailed description in conjunction with the associated drawings, which are described briefly below.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in this specification and constitute a part of it, illustrate several embodiments consistent with the disclosure. Together with the description, the drawings serve to explain the principles of the disclosure.

In the drawings:

FIG. 1A is an illustration of a polypeptide 100 according to some embodiments.

FIG. 1B schematically illustrates a method 150 for preparing two samples for performing mass spectrometry analysis on polypeptide 100 according to some embodiments.

FIG. 1C illustrates a preparation method 160 that utilizes a reduction scheme without an IdeS digestion, according to some embodiments.

FIG. 1D shows a schematic of a polypeptide to illustrate other examples of the middle portion according to some embodiments.

FIG. 2 is a flowchart of a method 200 for sequencing the polypeptides based on the products of the preparation methods according to some embodiments.

FIG. 3 is an illustration of the result of sequencing fully reduced polypeptide subunits according to some embodiments.

FIG. 4 is an illustration of the result of sequencing partially reduced polypeptide subunits according to some embodiments.

FIGS. 5 and 6 illustrate the effect that an intrachain disulfide bond may have on the dissociation of a polypeptide according to some embodiments.

FIG. 7 is a schematic representation of dissociation of three polypeptides 710, 720, and 730 according to some embodiments.

FIG. 8A is an alternative schematic representation of an intrachain disulfide bond according to an embodiment.

FIG. 8B shows a structure where peptide backbone is linked using a cross-linking agent 810.

FIG. 8C illustrates a reaction between a cross-liking agent 820 and polypeptide 830 according to some embodiments.

FIG. 8D shows an exemplary reaction according to such embodiments in which the reaction between polypeptide 830 and metal 850 forms a metal-polypeptide complex 860.

FIG. 8E illustrates formation of an extra link according to some embodiments.

FIG. 9 shows a flow chart for a method 900 for utilizing a chemical reaction with an agent to improve the top-down sequence coverage according to some embodiments.

FIG. 10 shows a flow chart for a method 1000 for performing a physicochemical interaction to achieve an improvement in top-down sequence coverage according to some embodiments.

FIG. 11 shows a liquid chromatography chromatogram showing the separation of two components and the mass analysis of each component.

### DETAILED DESCRIPTION

It will be appreciated that for clarity, the following discussion will explicate various aspects of embodiments of the applicant's teachings, while omitting certain specific details wherever convenient or appropriate to do so. For example, discussion of like or analogous features in alternative embodiments may be somewhat abbreviated. Well-known ideas or concepts may also for brevity not be discussed in any great detail. The skilled person will recognize that some embodiments of the applicant's teachings may not require certain of the specifically described details in every implementation, which are set forth herein only to provide a thorough understanding of the embodiments. Similarly, it will be apparent that the described embodiments may be susceptible to alteration or variation according to common general knowledge without departing from the scope of the disclosure. The following detailed description of embodiments is not to be regarded as limiting the scope of the applicant's teachings in any manner.

The following detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

In this disclosure, the following terms may be used with the following corresponding definitions.

Tandem mass spectrometry (MS/MS) is a method in which the mass spectrometry analysis maybe performed in multiple stages, for example, starting with isolating precursor ions, which may subsequently undergo an ion reaction or fragmentation, and then detecting the resulting products in a second mass spectrometry experiment. The same mass analyzer may be used to perform both experiments or multiple mass analyzers may be positioned in tandem to perform different experiments.

Fragmentation is a type of an ion reaction, where the products of the reaction include fragments of the ion.

A polypeptide is a polymer of amino acids joined together by peptide bonds. In some cases a number of polypeptides may be connected to each other either covalently, e.g., via disulfide bonds, or non-covalently, to form a larger polypeptide. In some cases, each of these polypeptides may be considered subunits of the larger polypeptide.

A polypeptide sequence, or a sequence, is a sequence of amino acids in a particular order stretching from one terminal to another terminal in the polypeptide. In the case of a complex polypeptide containing multiple polypeptide chains, the sequence may include the sequence of each polypeptide chain.

The term protein may be used interchangeably with polypeptide and may refer to a large polypeptide of an atomic mass that is greater than 10 kDa.

Bottom-up mass spectrometry is an MS sequencing approach in which intact proteins are digested into peptides prior to introduction into the mass spectrometer for sequencing.

Top-down mass spectrometry is an MS approach in which the protein is introduced directly to the mass spectrometer for sequencing, without being digested first.

Middle-down mass spectrometry is an MS approach based on a limited proteolytic digestion of the protein to generate larger polypeptides with atomic masses around 20 kDa before those polypeptides are introduced to the mass spectrometer for sequencing.

Collision-induced dissociation (CID) is a mass spectrometry technique that induces fragmentation of ions of interest in gas phase by depositing energy into the ions via imparting ions against a bath of gas.

Electron capture dissociation (ECD) is a fragmentation technique in which a polypeptide ion reacts with a free low energy electron, leading to electron capture, energy release, and in some cases dissociation of the ion.

Electron-transfer dissociation (ETD) is a fragmentation technique in which a polypeptide cation reacts with a reagent anion, followed by an electron transfer those results in a fragmentation process similar to ECD.

UV Photodissociation (UVPD) is method in MS in which ions are energized via high-energy photons, resulting in ion fragmentation.

A terminal portion of a polypeptide may include a portion adjacent to the N or C terminal of the polypeptide.

A middle portion of a polypeptide may include a portion starting about 50 amino acids away from a terminal or, when the polypeptide includes an intrachain disulfide bond or other linkage, a portion extending from an inner end (away from a terminal) of the intrachain disulfide bond or linkage.

Full reduction refers to removing essentially all interchain and intrachain disulfide bonds of a polypeptide.

Partial reduction refers to removing essentially all interchain disulfide bonds while retaining essentially all intrachain disulfide bonds of a polypeptide.

Next, proceeding to the detailed description of various embodiments, in recent years top-down and middle-down MS are gaining more traction. One exemplary area of interest is sequence analysis of monoclonal antibody (mAb). For certain mAb assays, it is often desirable to have close to 100% sequence coverage. Traditional bottom-up approaches with multiple proteases may deliver on this need, but may be time consuming and error prone. Existing top-down and middle-down approaches, on the other hand, may only result in partial sequence coverage mostly due to poor sequence coverage in the middle portion of the protein. Some embodiments are aimed at improving this situation.

Typically, CID fragmentation may be employed for top-down and middle-down analysis. However, sequencing approaches based on CID fragmentation are known to suffer from incomplete sequence coverage owing to the rather preferential cleavage of certain sites.

Other fragmentation techniques, such as ECD, ETD and UVPD, may be used to perform top-down and middle-down analysis and may yield better sequence coverage.

Some of the improved techniques according to various embodiments are described in relation to the following figures.

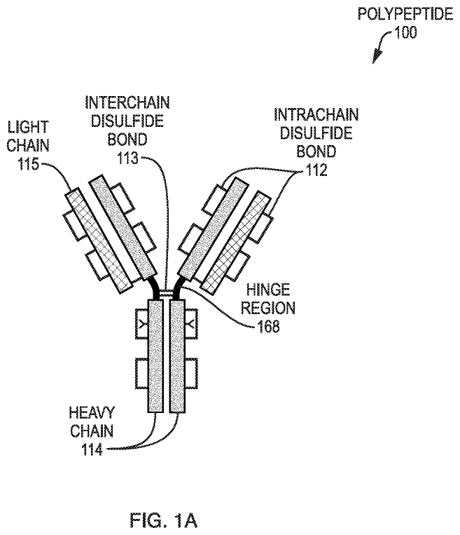

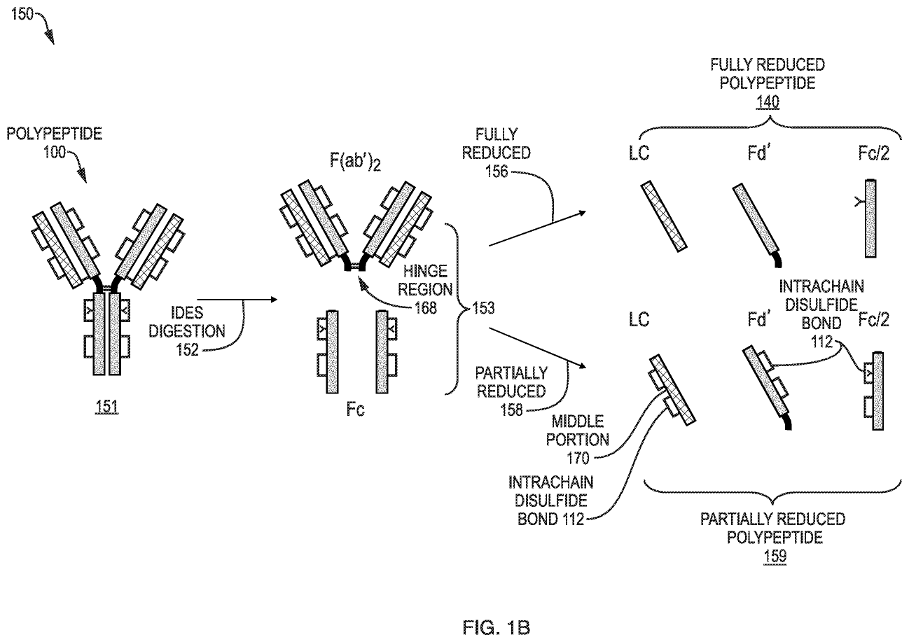

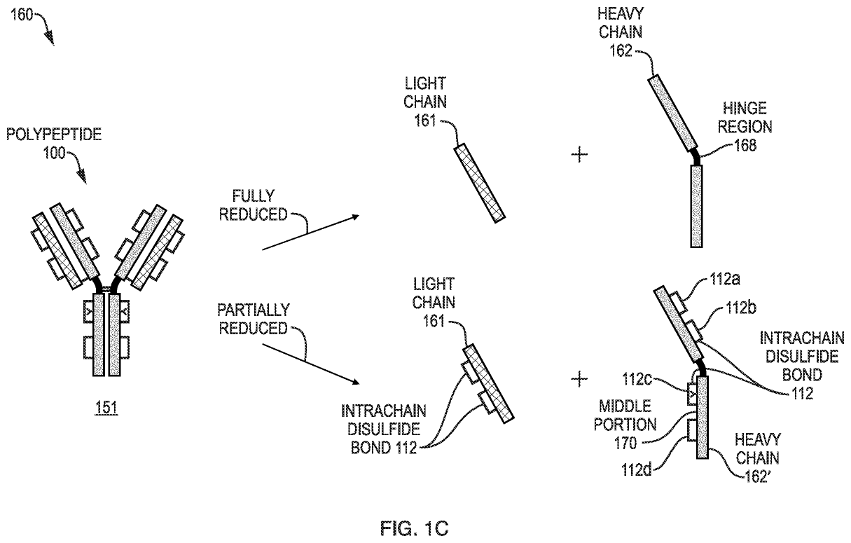

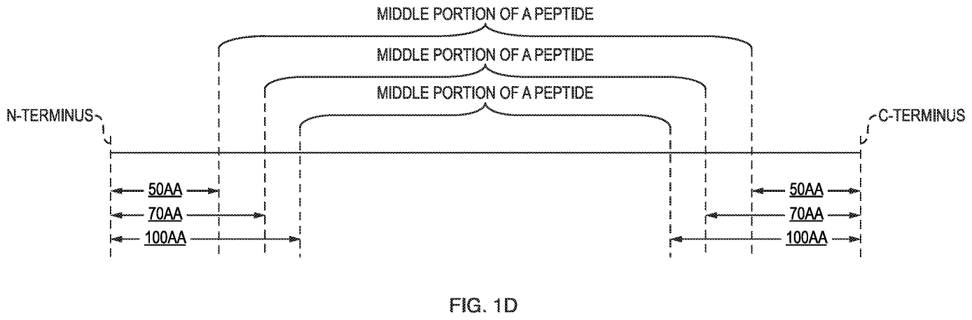

FIG. 1A is an illustration of a polypeptide 100 according to some embodiments. Polypeptide 100 may be, for example, an mAb. As shown in FIG. 1A, polypeptide 100 may include multiple chains of polypeptides such as heavy chains 114 and light chains 115. Moreover, polypeptide 100 may include a number of intrachain disulfide bonds 112 and a number of interchain disulfide bonds 113. As illustrated, an intrachain disulfide bond 112 may form a linkage between amino acids within the same chain while an interchain disulfide bond 113 may form a linkage between different polypeptide chains. Moreover, polypeptide 100 may also include hinge regions 168 that connects its antigen binding fragment (Fab) with its crystallizable fragment (Fc).

FIG. 1B schematically illustrates a method 150 for preparing two samples for performing mass spectrometry analysis on polypeptide 100 according to some embodiments. Method 150 illustrates such preparation for a middle-down analysis of antibodies utilizing technologies such as ECD, ETD and UVPD of mAbs and involving a step of limited proteolytic digestion as further described below.

As shown in FIG. 1B, method 150 starts at step 151, wherein a stock solution of an antibody sample is obtained. This stock solution may then undergo an optional step of limited proteolytic digestion (e.g., an IdeS digestion; (step 152)), which may transform the intact antibody sample into a digested sample 153, including a few subunit types. The IdeS (Immunoglobulin-degrading enzyme from Streptococcus pyogenes) is an engineered recombinant protease overexpressed in Escherichia coli. The IdeS digestion may specifically cleave IgG molecules below hinge region 168 to yield a digested sample 153, which may include F(ab′)2 (Fragment antigen binding) and Fc (crystallizable fragment), as shown in FIG. 1B.

Next, at step 156, a first subsample may be taken from digested sample 153 and exposed to a process of full reduction to generate a first set of subunits 140. In some embodiments, during the full reduction, approximately all interchain and interchain disulfide bonds are removed. In some other embodiments, a high percentage of these bonds may be removed, and less than 10% of the subsample may contain one or more disulfide bonds. Some detailed techniques of performing the full reduction are described below.

As shown in FIG. 1B, first set of subunits 140, which results from the full reduction of the first subsample, may include subunits that comprise fully reduced polypeptides such as LC (light chain), Fd′ (heavy chain part of Fab), and Fc/2.

Alternatively, at step 158, a second subsample may be taken from digested sample 153 and exposed to a process of partial reduction to generate a second set of subunits 159. In some embodiments, during the partial reduction, essentially all or a high percentage of interchain disulfide bonds 113 may be removed, leaving essentially all or a high percentage of intrachain disulfide bonds 112 intact. Some further detailed techniques of performing the partial reduction are described below. As also shown in FIG. 1B, second set of subunits 159 may include subunits such as LC, Fd′, and Fc/2 with intact intrachain disulfide bonds.

Some embodiments utilize preparation methods that include reduction schemes without an IdeS digestion, therefore resulting in sets of subunits that include light chains and heavy chains. FIG. 1C illustrates a preparation method 160 that utilizes such a reduction scheme without an IdeS digestion, according to some embodiments. In method 160, unlike in method 150, the starting sample may not undergo the IdeS digestion. Instead, the first subsample and the second subsample may be taken from the starting sample and respectively exposed to the processes of full reduction and partial reduction, as described above. As a result, the generated sets of subunits may include not only a significant percentage of light chains 161, but also a significant percentage of heavy chains 162, as shown in FIG. 1C.

Some embodiments enable sequencing different portions of a polypeptide such as the middle portion. A middle portion 170 of a polypeptide, in the case of a light chain shown in FIGS. 1B and 1C, may be defined as the portion of the polypeptide between the two intrachain disulfide bonds. In the case of a heavy chain such as heavy chain 162′, (which results from the partial reduction) shown in FIG. 1C, middle portion 170 may be defined as the portion of the polypeptide between the two outer intrachain disulfide bonds 112a and 112d, but excluding portions that are enclosed by intrachain disulfide bonds 112b and 112c.

FIG. 1D shows a schematic of a polypeptide to illustrate other examples of the middle portion according to some embodiments. In FIG. 1D, the polypeptide is shown as the straight line connecting an N-terminus to a C-terminus. As shown in FIG. 1D, in various embodiments, the middle portion of the polypeptide may be defined as the portion of the polypeptide away from a terminus by 50, 70, or 100 amino acids (AA).

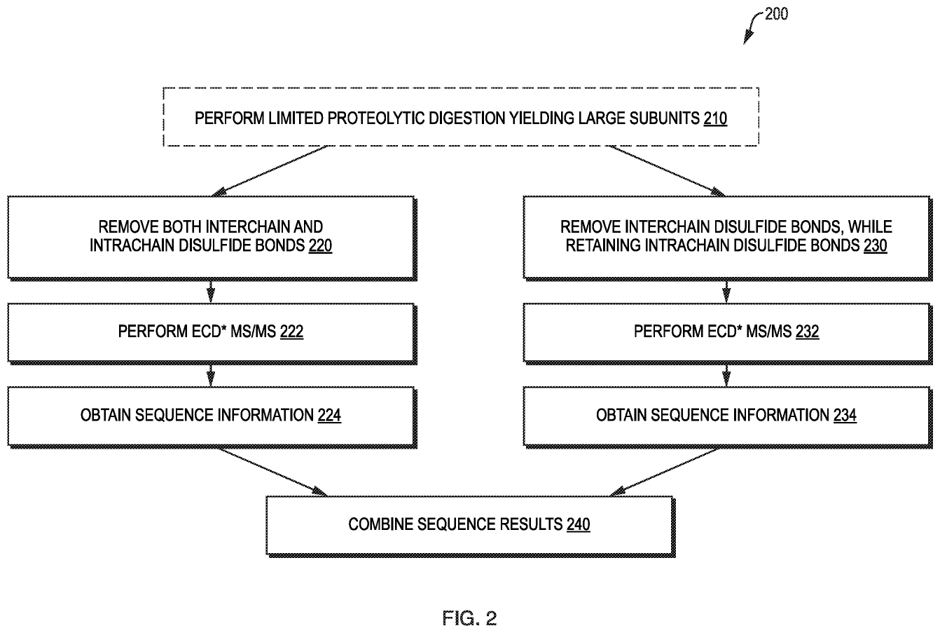

In some embodiments, the products of the above discussed preparation methods may be then undergoing MS analyses for sequencing the polypeptide. FIG. 2 is a flowchart of a method 200 for sequencing the polypeptides based on the products of the preparation methods according to some embodiments.

At step 210, the limited proteolytic digestion is performed on a sample, such as a sample of a protein, which may yield a sample of large polypeptides. This process corresponds to step 152 of method 150 in FIG. 1B. The method then bifurcates and divides into two distinct paths for two different sets of operations performed on two subsamples of the sample.

Considering the first path, at step 220, in the first subsample, both interchain and intrachain disulfide bonds are removed and the first set of subunits are obtained. This step corresponds to step 156 of method 150 in FIG. 1B.

At step 222, an ECD* MS/MS is performed to sequence the first set of subunits. As a result, at step 224, a first sequence corresponding to the first set of subunits is obtained. The asterix in ECD* indicates that instead of ECD MS/MS other techniques may be utilized such as ETD or UVPD MS/MS.

Next, considering the second path, at step 230, in a second subsample of the polypeptides interchain disulfide bonds are removed while intrachain disulfide bonds are retained and thereby the second set of subunits are obtained. This process corresponds to step 158 of method 150 in FIG. 1B.

At step 232 an ECD* MS/MS is performed to sequence the second set of subunits. As a result, at step 234, a second sequence corresponding to the second set of subunits is obtained.

At step 240, the first sequence and the second sequence are combined to obtain a combined sequence corresponding to the protein in the sample.

It should be noted that method 200 may start with a protein or a portion of a protein or a large polypeptide, and the process may still follow the same steps of preparing reduced subunits, acquiring sequences of these subunits, and combining the sequences to obtain a combined sequence.

The processes for removing interchain and intrachain disulfide bonds may be performed according to the following steps for the example of preparation of IDES subunits of NIST mAb with (a) fully reduced disulfide bonds in the full reduction process and (b) partially reduced disulfide bonds in the partial reduction process.

### (a) Preparation of Fully Reduced NIST mAb IdeS Subunits:

a. Mix 40 μL of NIST mAb Stock Solution (10 μg/μL) with 360 μL water to produce NIST mAb (1 μg/μL).
b. To 50 μL NIST mAb (1 μg/μL), add 2 μL of IdeS enzyme (25 units/μL) to and incubate mixture at 37 °C for 2 hours.
c. To 50 μL of digested sample, add 157 μL of denaturing reagent (7.2 Guanidine in 50 mM Tris, pH 7-8) and vortex.
d. To mixture, add 23 μL 1 M DTT and incubate for 30 mins at 60 °C.

### (b) Preparation of Partially Reduced NIST mAb Subunits:

e. Mix 10 μL of NIST mAb Stock Solution (10 μg/μL, 67 pmol/μL) with 119.3 μL of 0.01M PBS buffer to yield NIST mAb dilution (5 pmol/μL).
f. Add 4 μL of IdeS enzyme (25 units/μL) and incubate mixture at 37 °C for 60 minutes.
g. After incubation, add 10 μL Reducing agent (TCEP, 50 mM) to the solution. Incubate 37 °C for 30 minutes.
h. Add 4.1 μL of 243 mM Iodoacetamide and incubate for 30 mins at 37 °C in dark.

The above techniques may result in a high yield for producing partially reduced or fully reduced subunits. For example, in some embodiments, the yield rates are greater than 90%.

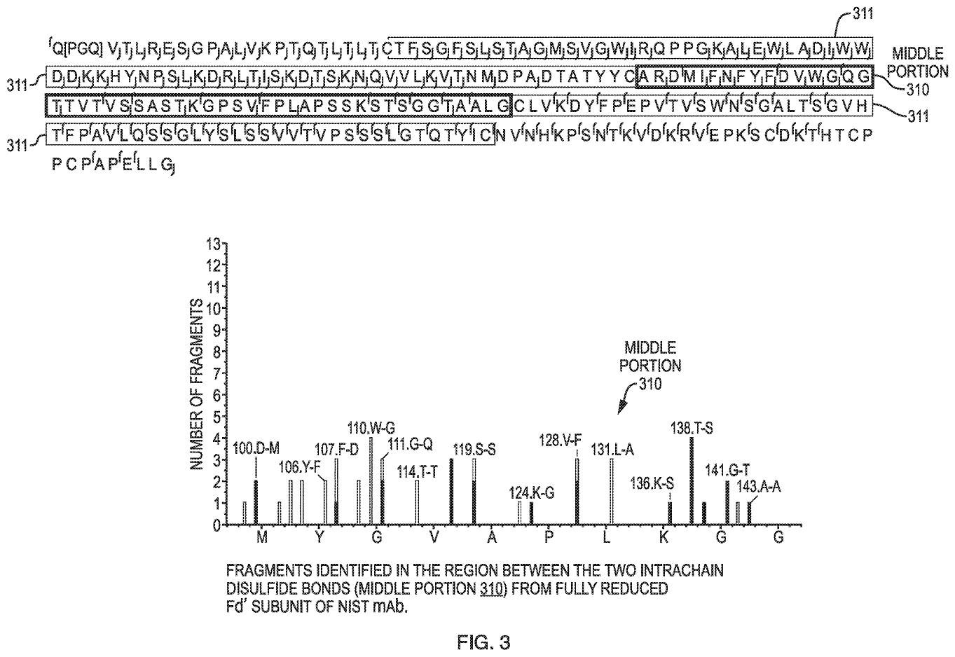

FIG. 3 is an illustration of the result of sequencing fully reduced polypeptide subunit Fd′ (c.f. FIG. 1B) after the full reduction according to the embodiments described at steps 220, 222, and 224 of method 200 (c.f. FIG. 2). The top section of FIG. 3 shows the identified amino acid sequence exemplary subunit NIST mAb with cleavage sites, in fully reduced experiment according to some embodiments. The light boxed region 311 at the top indicates amino acids belonging to a portion of the sequence that would have been enclosed by an intrachain disulfide bond before full reduction. This plot indicates good sequence coverage of region 311 but poor sequence coverage in the middle portion 310 (dark boxed region) of the polypeptide subunit.

The bottom section of FIG. 3 is a plot of the number of fragment ions identified in the middle portion 310, the region between the two intrachain disulfide bonds, at different cleavage sites of the fully reduced Fd′ subunit accordingly. This plot indicates poor fragment identification in the middle portion 310 of the polypeptide subunit in the dark boxed region at the top section of FIG. 3.

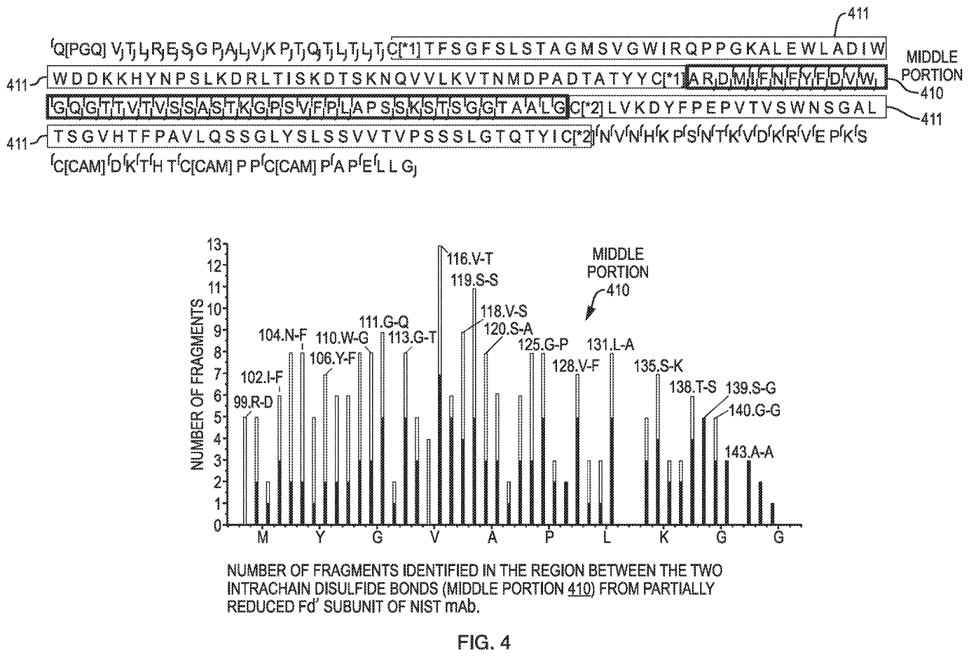

FIG. 4 is an illustration of the result of the sequencing partially reduced polypeptide subunit Fd′ (c.f. FIG. 1B) after partial reduction according to the embodiments described in steps of 230, 232, and 234 of method 200 (c.f. FIG. 2). The top section of FIG. 4 shows the identified amino acid sequence exemplary subunit NIST mAb with cleavage sites, in a partially reduced experiment according to some embodiments. The dark boxed region at the top section of FIG. 4 indicates amino acids belonging to a middle portion 410, a portion of the sequence that is not enclosed by an intrachain disulfide bond. The light boxed region 411 at the top of the figure indicates amino acids belonging to a portion of the sequence enclosed by an intrachain disulfide bond. The plot also indicates poorer sequence coverage in the region 411 covered by intrachain disulfide bonds.

The bottom section of FIG. 4 is an illustration of the number of fragment ions identified in middle portion 410, the region between the two intrachain disulfide bonds at different cleavage sites of partially reduced Fd′ subunit accordingly. As shown in the figure, the plot indicates good fragment identification of the middle portion of the polypeptide subunit.

Accordingly, as demonstrated in FIGS. 3 and 4, combining the sequences of fully reduced and partially reduced subunits may provide an improved sequence coverage of a polypeptide according to various embodiments. In particular, various embodiments provide methods with improved sequence coverage of the middle portion of the polypeptide, and as a result, a more complete sequence of the polypeptide as a whole.

In some embodiments, a fragmentation technique may be utilized to determine an amino acid sequence. The fragmentation technique may include an electron activated dissociation, the above mentioned ECD, ETD, or UVPD.

In some embodiments, fully reduced subunits and partially reduced subunits are produced from portions of the same original sample of the protein. Accordingly, the fully reduced sample and the partially reduced sample are subsamples of the same sample.

In some embodiments, the sequenced protein is an antibody, which may be a monoclonal antibody (mAb), a bispecific antibody (bsAb), or an antibody drug conjugate (ADC). The disclosed embodiments may apply to these cases and provide improved sequence coverage.

In some embodiments, removing a disulfide bond is performed by using a chemical agent. Examples of suitable chemical agents may include DTT (Dithiothreitol), TCEP (tris (2-carboxyethyl) phosphine) or mercaptoethanol.

In some embodiments, removing a disulfide bond is performed electrochemically.

In some embodiments an enzymatic digestion of the sample is performed prior to removing a disulfide bond. In some embodiments, an enzyme performing the enzymatic digestion may be IdeS, IgdE, IdeZ, Kgp, or SpeB

It should be noted that the mAb example given in some of the embodiments is for illustration purposes and the method of these embodiments may be applied to different types of proteins, portions of proteins, or polypeptides.

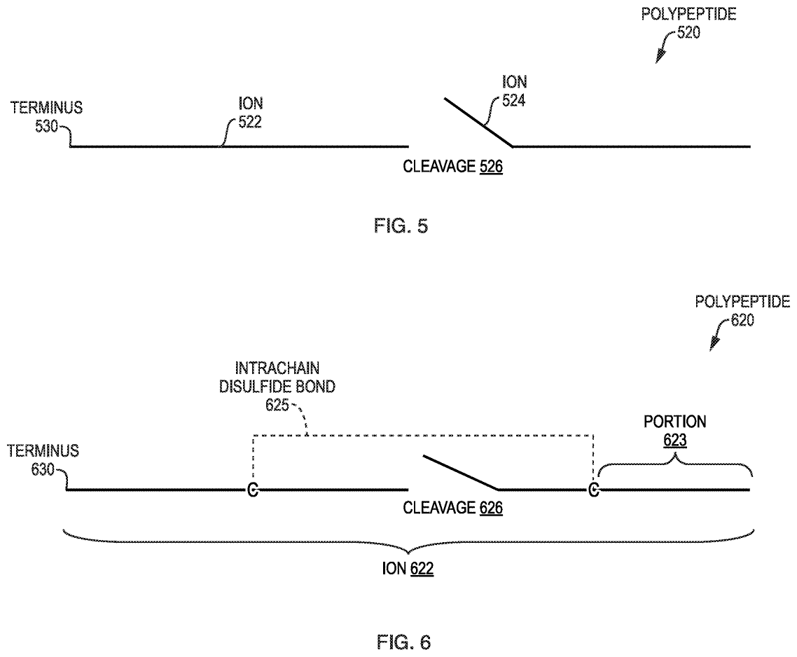

FIGS. 5 and 6 illustrate the effect that an intrachain disulfide bond may have on the dissociation of a polypeptide according to some embodiments.

More specifically, FIG. 5 illustrates a polypeptide 520 that has been dissociated at a cleavage 526. As a result, two product ions 522 and 524 are produced.

FIG. 6, on the other hand, illustrates a polypeptide 620 undergoing a fragmentation according to some other embodiments. Polypeptide 620 includes a backbone ion 622 in which a cleavage 626 is generated. Polypeptide 620 includes an intrachain disulfide bond 625 that links the two portions of backbone ion 622 separated by cleavage 626. Due to the presence of this linkage by intrachain disulfide bond 625, after the dissociation, a single product ion 622 is produced. According to some embodiments, the linked polypeptide provides improved sequence coverage of the polypeptide in the portion of the polypeptide indicated as portion 623 in FIG. 6.

Accordingly, since such an extra linkage afforded by intrachain disulfide-bonded polypeptide is beneficial, in some additional embodiments, different techniques are used to achieve a similar advantage.

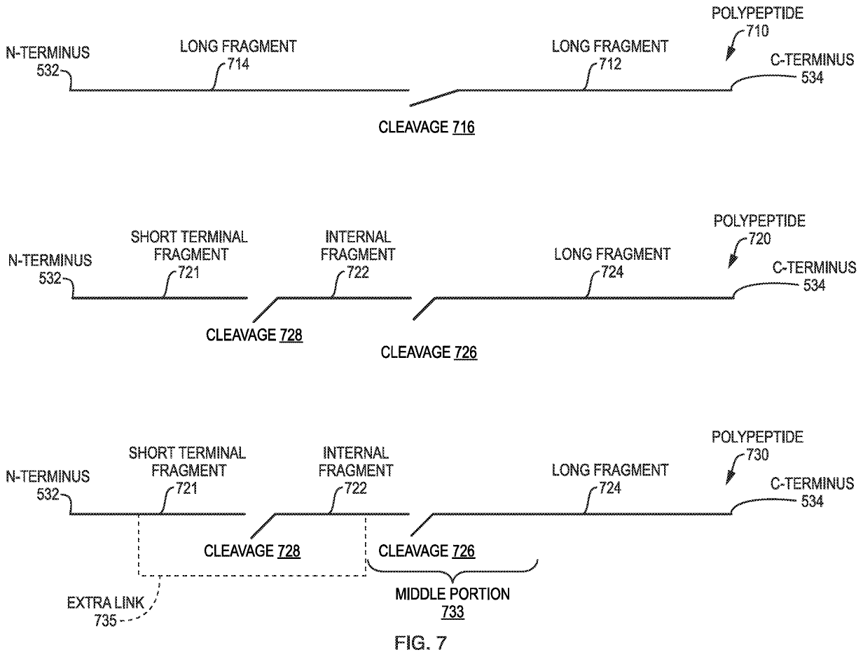

FIG. 7 is a schematic representation of dissociation of three polypeptides 710, 720, and 730, as further detailed below and according to some embodiments. More particularly, polypeptide 710 is a linear polypeptide that has undergone a single dissociation via cleavage 716. Polypeptide 720, on the other hand, is a linear polypeptide that has undergone multiple dissociations via cleavages 726 and 728. Finally, polypeptide 730 is a polypeptide that is nonlinear due to the existence of an extra link 735, and has similarly undergone multiple dissociations via cleavages 726 and 728.

Polypeptides 710 and 720 illustrate how multiple fragmentation events may redistribute the total ion intensity from the middle portion of the polypeptide towards the outer portion of the polypeptide. In particular, in polypeptide 710, cleavage 716 has generated two fragments 712 and 714. Both fragments 712 and 714 are long fragments.

In polypeptide 720, on the other hand, two cleavages 726 and 728 have generated a long fragment 724, a short terminal fragment 721, and a short internal fragment 722 due to secondary fragmentation.

According to some embodiments, polypeptide 730 having a link, such as extra link 735, may essentially preserve the longer fragment from a secondary fragmentation. This will improve the sequence coverage in an MS/MS sequencing process accordingly since the extra link provides a linkage between short terminal fragment 721 and internal fragment 722. According to these embodiments, providing the extra link improves sequence coverage in middle portion 733.

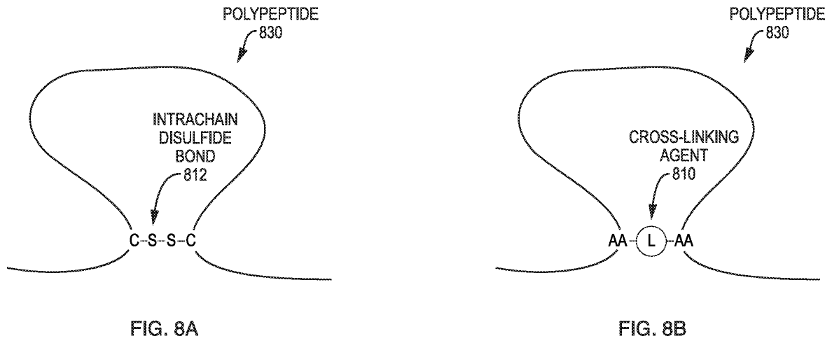

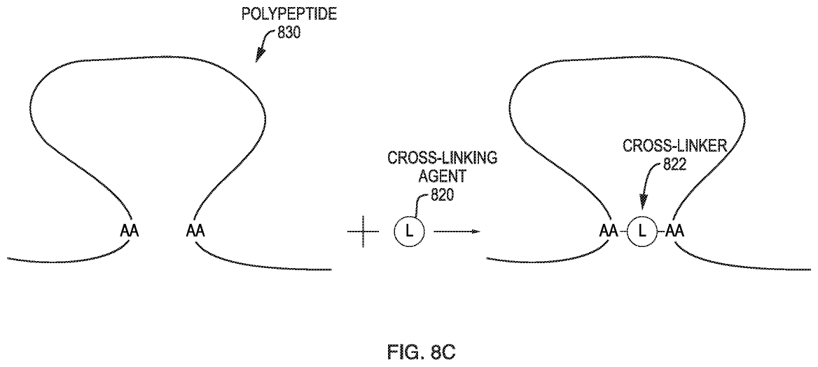

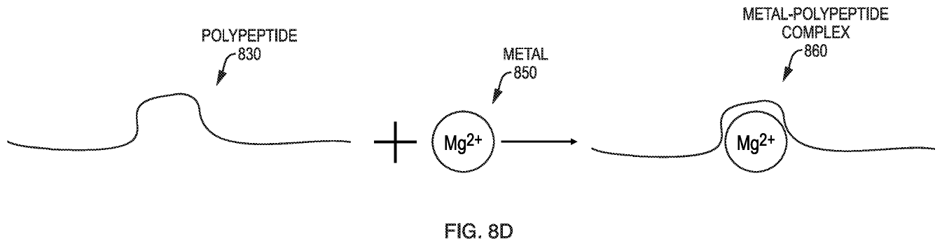

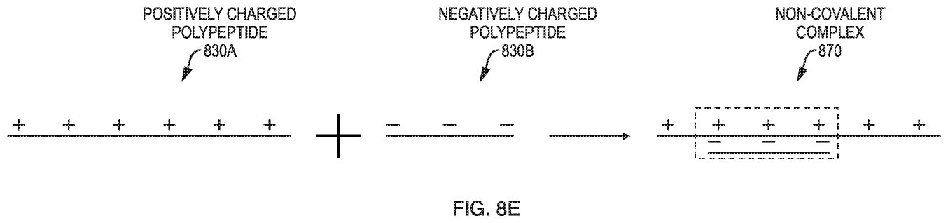

FIG. 8A is an alternative schematic representation of an intrachain disulfide bond according to an embodiment. In this embodiment, the distance between the cysteine residues forming the disulfide bond is shown closer, which is more realistic compared to the schematic in FIG. 6. In this more realistic illustration, the peptide backbone is shown to form a knot. This representation helps illustrate embodiments in which a link between the peptide backbone is introduced artificially as described below.

FIG. 8B shows a structure where peptide backbone is linked using a cross-linking agent 810. In this embodiment, cross-linking agent 810 is designed to react with specific groups of amino acid residues. In some embodiments, Lysine is used as a target amino acid given its relatively high prevalence of ~6%. In some embodiments N-hydroxysuccinimidyl or sulfosuccinimidyl esters have been reported as cross-linking agent, wherein the lysine is targeted.

FIG. 8C illustrates a reaction between a cross-liking agent 820 and polypeptide 830, which provides a cross-link 822. Following this process, the resulting analyte is similar to a polypeptide with an intrachain disulfide bond. The cross-linker improves sequence coverage of the polypeptide accordingly.

In this case, a first mass spectrometry analysis may be performed on a subsample without a reaction and another mass spectrometry analysis may be performed on a subsample with a reaction. Alternatively, only the subsample with a reaction may be analyzed, which improves sequence coverage in a certain region of interest according to some embodiments. These embodiments may not require the sample to have disulfide bonds and may be applied to other proteins and polypeptides.

In some embodiments, the reactant may be a metal cation and the polypeptide may have a site that accepts the reactant. FIG. 8D shows an exemplary reaction according to such embodiments in which the reaction between polypeptide 830 and metal 850 forms a metal-polypeptide complex 860. Metals and polypeptides are known to form a donor-acceptor bond. Other types of bonds, such as ionic bonds, are also possible. The formed complex then may be stabilized and therefore may be immune to a secondary fragmentation in the specific region of a polypeptide sequence where the complex is forming. Accordingly, this process may be utilized to achieve the goal of improved sequence coverage according to these embodiments.

In some embodiments, illustrated in FIG. 8E, the extra link is afforded by a gas phase reaction such as, for example, in a reaction between an analyte such as a positively charged polypeptide 830A and a reactant such as a negatively charged polypeptide 830B. In these embodiments, the two ions maybe attracted to each other and form a non-covalent complex 870. Such a reaction may be performed in an ion trap with a capability for mutual trapping of positive and negative ions.

In some embodiments, a precursor ion corresponding to an analyte ion is selected first and delivered to an ion-ion reaction device. The reactant ion is also selected and also delivered to the ion-ion reaction device. The reactant and analyte ions are then allowed to mix and interact in the reaction chamber such that ion-ion reaction products are formed. Optionally, the process may include selection of a specific product that corresponds to a complex formed by one analyte, and single or multiple reactant ions. This complex is then further subjected to fragmentation and the resulting products are analyzed. Similar to a cross-linking agent, this reaction doesn't require the analyte polypeptide to contain a disulfide bond. Unlike the disulfide bond or cross-link case, however, in this case the site of the reaction may be random. However, if the analyte polypeptide size is substantially larger than the reactant polypeptide, a sufficient portion of the analyte polypeptide may be unobstructed for fragmentation reactions, and accordingly, secondary fragmentation events may be prevented in the area where the complex is formed.

In some embodiments, it may be advantageous to perform the fragmentation using an electron capture mechanism since a positive charge is needed for such a reaction. Given the fact that the reactant ion is negatively charged, the probability of cleavage of the reactant ion may be greatly diminished in cases where the fragmentation methods may require the electron for the reaction. In the region where the reactant ion interacts with the analyte ion, the total charge may be zero, and hence, that portion of the complex will be neutral. This may also greatly diminish the reaction efficiency, further preventing unwanted fragmentation of the reactant polypeptide. Because of this phenomenon, generally, there may be two series of products detected: one corresponding to the fragments observed following the fragmentation of an unreacted analyte ion and another in which the fragment also contains a reactant polypeptide modification. Other products, such as fragments of the reactant polypeptide may be less likely in this case. Since there are only two series of fragments formed under this process, the signal dilution may be minimal and an improved sequencing of the middle portion of the polypeptide may be realized in this scheme.

Some embodiments may be used to sequence other biopolymers such as polynucleotides. For example, single-strand DNA and RNA polymer molecules may consist of many nucleotide bases and suffer from similar issues in sequencing the middle portion of the polynucleotide, owing to secondary fragmentation. These molecules may be preferentially analyzed in negative mode and therefore the reaction with a positive reactant may be suitable. Such a reactant may be a positively charged polypeptide.

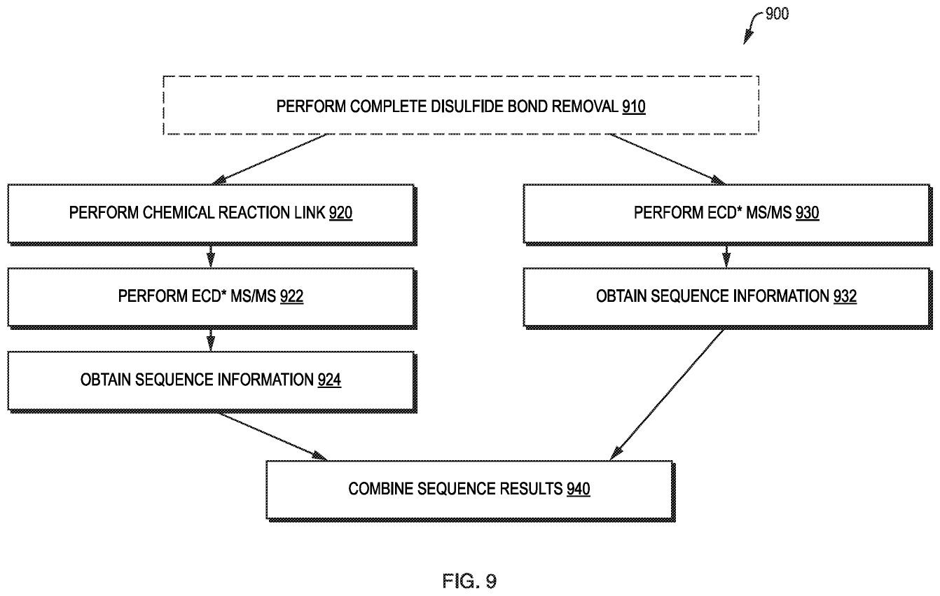

FIG. 9 shows a flow chart for a method 900 for utilizing a chemical reaction with an agent to improve the top-down sequence coverage according to some embodiments. The chemical reaction may stitch together different portions of the polypeptide.

In some embodiments, the chemical reaction is performed in liquid phase. In some embodiments the reagent may be a metal cation. In some embodiments the metal cation forms other types of bonds such as donor-acceptor bond or ionic bond. In some embodiments a cross-linking agent may be used, which may form a covalent bridge between two amino acids in the polypeptide.

Method 900 starts with an optional step of performing complete disulfide bond removal of a polypeptide at step 910. The process then bifurcates and divides into two distinct paths.

In one path, at step 920, method 900 utilizes a chemical reaction to link a portion of polypeptide. At step 922, ECD* MS/MS analysis is performed on the resulting subunits. At step 924 a first sequence of the subunits is obtained.

In another path, at step 930, ECD* MS/MS analysis is performed on the subunits without using chemical reaction to link a polypeptide. At step 932 a second sequence of these subunits is obtained.

At step 940, the first sequence and the second sequence are combined to obtain a combined sequence coverage of the polypeptide. The combined sequence may provide improved coverage of the sequence of the polypeptide according to these embodiments.

In some embodiments, a similar goal to that of the embodiments of FIG. 9 is achieved by performing a physicochemical interaction to link a portion of the polypeptide, which may provide a secondary link connecting multiple fragments of the polypeptide together. Such a physicochemical interaction to link a portion of the polypeptide may be achieved by an ion-ion reaction of an analyte and ion reactant.

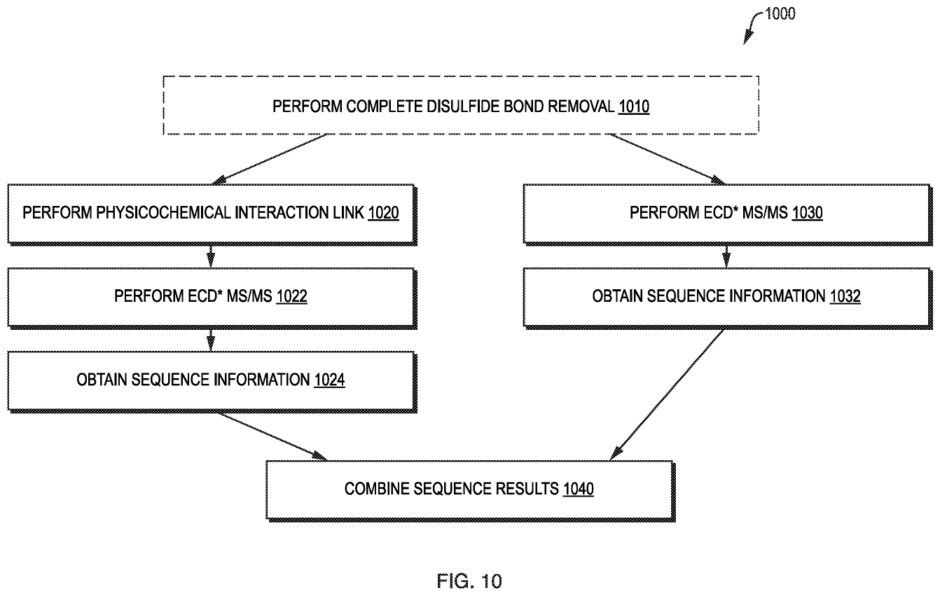

In particular, FIG. 10 shows a flow chart for a method 1000 for performing a physicochemical interaction to achieve an improvement in top-down sequence coverage according to some embodiments. The physiochemical interaction may link different portions of the polypeptide.

Method 1000 starts with an optional step of performing complete disulfide bond removal of a polypeptide at step 1010. The process then bifurcates and divides into two distinct paths.

In one path, at step 1020, a physicochemical interaction is performed to link a portion of the polypeptide. At step 1022, ECD* MS/MS analysis is performed on the resulting subunits. At step 1024 a first sequence of the subunits is obtained.

In another path, at step 1030, ECD* MS/MS analysis is performed on the subunits without performing a physicochemical interaction to link a portion of the polypeptide. At step 1032 a second sequence of these subunits is obtained.

At step 1040, the first sequence and the second sequence are combined to obtain a combined sequence of the polypeptide. The combined sequence may provide improved coverage of the sequence of the polypeptide according to these embodiments.

In certain embodiments, the denaturation and reduction of a monoclonal antibody (mAb) to produce a fully reduced and disulfide-linked light chain (LC) in the same sample can be made. The concentration of guanidine hydrochloride (Gnd), the temperature and duration of incubation were key factors in determining the ratio of the fully reduced vs. disulfide-linked subunits. A final concentration of 2M Gnd and an incubation temperature and time of 45 °C and 30 minutes, respectively, led to the co-formation of the fully reduced and disulfide-linked LC subunits of NISTmAb. The two species can be separated (e.g., chromatographically such as liquid chromatography) and targeted for fragmentation in accordance with the present teachings in a single high resolution MRM experiment. The analysis of the combined result of the two species led to ultrahigh sequence coverage (>85%) in a single injection.

An aliquot of 10 μg/μL NISTmAb was mixed with guanidine hydrochloride (Gnd) and dithiothreitol (DTT) at a final concentration of 2M and 50 mM, respectively. The mixture was incubated at 45 °C for 30 minutes. 4 μL of the final solution (~4 μg) was injected for liquid chromatography mass spectrometry (LCMS) analysis immediately following the completion of the incubation.

The subunits of NISTmAb were separated using an ACQUITY UPLC Protein BEH C4 column (2.1×50 mm, 1.7 μm, 300 Å, Waters). The liquid chromatography gradients used for the subunit separation are shown in Table 1. A flow rate of 0.3 mL/min was used for all of the liquid chromatography runs. The column was kept at 60 °C in the column oven of an ExionLC system (SCIEX). Mobile phase A was 0.1% formic acid in water and mobile phase B was 0.10% formic acid in acetonitrile. The initial concentration of the gradient consisted of 80% A and 20% B, ramping down gradually to a 10% A and 90% B at 15 min and then back to 80% A and 20% B at 20 min.

TABLE 1
Liquid chromatography gradient for subunit separation

| Time (min) | A(%) | B(%) |
|---|---|---|
| Initial | 80 | 20 |
| 2 | 80 | 20 |
| 14 | 60 | 40 |
| 15 | 10 | 90 |
| 17 | 10 | 90 |
| 17.5 | 80 | 20 |
| 20 | 80 | 20 |

MS analysis was performed using the ZenoTOF 7600 system (SCIEX). 2 charge states of the fully reduced (24+ and 19+) and disulfide-linked (18+ and 15+) LC subunits were targeted for fragmentation in accordance with the present teachings in this work (e.g., electron based dissociation such as ECD*).

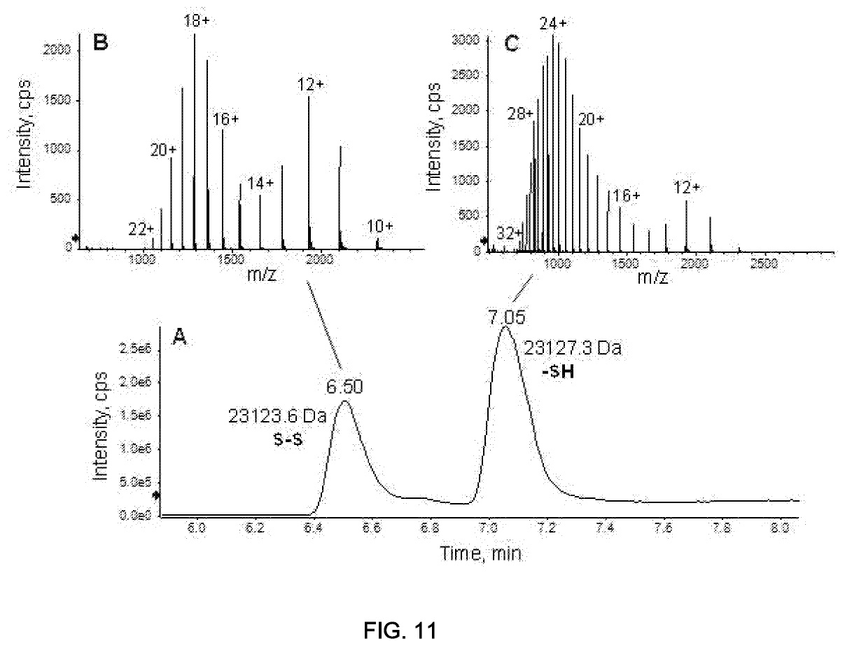

A final concentration of 2M Gnd and an incubation temperature and time of 45 °C and 30 minutes, respectively, led to the co-formation of the fully reduced and disulfide-linked LC subunits of NISTmAb in the same sample as shown in FIG. 11. These two forms of the LC subunit were able to be chromatographically resolved as shown in panel A of FIG. 11. The mass spectra of the two species identified are depicted in panels B and C of FIG. 11 and show a distinct difference in charge state distribution. The disulfide-linked LC subunit carried fewer charges on average compared to the fully reduced counterpart due to a lesser degree of sequence unfolding because of the presence of 2 intra-chain disulfide bonds. Automated protein deconvolution performed using Biologics Explorer software confirmed the masses of the fully reduced (23127.3 Da) and disulfide-linked (23123.6 Da) LC subunits. Two charge states from each form of the LC subunit were targeted for fragmentation in a single mass spectrometry experiment. The processing of this data separately or jointly led to confident disulfide bond mapping and ultrahigh sequence coverage of the initial peptide.

In some embodiments, the physicochemical interaction is performed via a gas phase reaction. The physicochemical interaction may be the result of an ion-ion reaction. In some embodiments, the reagent may be a polypeptide of an opposite charge.

In some embodiments, the analyte polypeptide mass is above 20 kDa. The sequence may then contain at least a portion of the middle of the protein.

Those having ordinary skill will appreciate that various changes may be made to the above embodiments without departing from the scope of the disclosure.

Although some aspects have been described in the context of a system or an apparatus, it is clear that these aspects may also represent a description of the corresponding method, where a block or device corresponds to a method step or a feature of a method step. Analogously, aspects described in the context of a method step also represent a description of a corresponding block or item or feature of a corresponding apparatus.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Also, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or.

Moreover, if these terms are used, a set may include one or more members, and a subset of a set may include one or more than one, including all, members of the set.

The disclosed compositions, systems, methods, and apparatus are not limited to any specific aspect or feature or combinations thereof, nor do the disclosed compositions, systems, methods, and apparatus require that any one or more specific advantages be present or problems be solved. Any theories of operation are to facilitate explanation, but the disclosed compositions, systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

1. A method for sequencing a polypeptide, the method comprising:
removing one or more interchain disulfide bonds and retaining essentially all intrachain disulfide bonds of a first sample of the polypeptide to generate a first set of subunits;
performing a first mass spectrometry (MS) analysis of the first set of subunits to determine a first amino acid sequence of the polypeptide, removing one or more interchain disulfide bonds and one or more intrachain disulfide bonds of a second sample of the polypeptide to generate a second set of subunits;
performing a second MS analysis of the second set of subunits to determine a second amino acid sequence of the polypeptide; and
combining the first amino acid sequence and the second amino acid sequence to determine a combined amino acid sequence of the polypeptide.

2. The method of claim 1, wherein a fragmentation process is utilized to determine an amino acid sequence.

3. The method of claim 2, wherein the fragmentation process is selected from one of electron activated dissociation and electron capture dissociation, ultraviolet photo-dissociation, and electron transfer dissociation.

4. The method of claim 1, wherein removing one or more interchain disulfide bonds includes removing essentially all interchain disulfide bonds.

5. The method of claim 1, wherein the second sample is the same as the first sample.

6. The method of claim 1, wherein the first sample and the second sample are subsamples of the same sample.

7. The method of claim 1, wherein the first sequence contains at least a section from a middle portion of the polypeptide.

8. The method of claim 1, wherein the second sequence contains at least a portion enclosed by intrachain disulfide bond.

9. The method of claim 1, wherein the polypeptide is a protein.

10. The method of claim 9, wherein the polypeptide is an antibody.

11. The method of claim 10, wherein the antibody is one of a monoclonal antibody (mAb), a bispecific antibody (bsAb), and an antibody drug conjugate (ADC).

12. The method of claim 1, wherein tandem mass spectrometry analysis is used for MS analysis.

13. The method of claim 1, wherein removing a disulfide bond is performed by using a chemical agent, and
optionally, wherein the chemical agent is selected from a group consisting of DTT (Dithiothreitol), TCEP (tris (2-carboxyethyl) phosphine) and mercaptoethanol.

14. (canceled)

15. The method of claim 1, wherein removing a disulfide bond is performed electrochemically.

16. The method of claim 15, wherein an enzymatic digestion of the sample is performed prior to removing a disulfide bond, and
optionally, wherein an enzyme performing the enzymatic digestion is one of IdeS, IgdE, IdeZ, Kgp, and SpeB.

17. (canceled)

18. The method of claim 1, wherein removing one or more interchain disulfide bonds of the first sample comprises removing essentially all interchain disulfide bonds.

19. The method of claim 1, wherein removing one or more interchain disulfide bonds and one or more intrachain disulfide bonds of the second sample comprises removing essentially all interchain and intrachain disulfide bonds.

20-33. (canceled)

34. A method for sequencing a middle portion of a polypeptide, the method comprising: removing essentially all interchain disulfide bonds and retaining essentially all intrachain disulfide bonds of a sample of the polypeptide to generate subunits, and performing mass spectrometry (MS) analysis of the subunits to determine an amino acid sequence of the middle portion of the polypeptide.

35. The method of claim 34, wherein the middle portion of the polypeptide spans a portion further than 50 amino acids from the polypeptide termini,
optionally, wherein the middle portion of the polypeptide spans a portion further than 70 amino acids from the polypeptide termini, and
optionally, wherein the middle portion of the polypeptide spans a portion further than 100 amino acids from the polypeptide termini.

36. (canceled)

37. (canceled)

38. A method for sequencing a polypeptide from a sample, the method comprising:
removing one or more interchain disulfide bonds and retaining essentially all intrachain disulfide bonds of a first portion of a sample of the polypeptide to generate a first set of subunits;
removing one or more interchain disulfide bonds and one or more intrachain disulfide bonds of a second portion of the sample of the polypeptide to generate a second set of subunits;
separating the first set of subunits from the second set of subunits;
performing a mass spectrometry (MS) analysis of the first set of subunits to determine a first amino acid sequence of the polypeptide;
performing a second MS analysis of the second set of subunits to determine a second amino acid sequence of the polypeptide; and
combining the first amino acid sequence and the second amino acid sequence to determine a combined amino acid sequence of the polypeptide.
